(19) United States
(12) Patent Application Publication
(10) Pub. No.: US 2025/0091106 A1
(43) Pub. Date: Mar. 20, 2025

(54) MYCOREMEDIATION OF CONTAMINANTS

(71) Applicant: MYCOCYCLE, INC., Bolingbrook, IL (US)

(72) Inventor: Joanne Rodriguez, Bolingbrook, IL (US)

(21) Appl. No.: 18/577,341

(22) PCT Filed: Nov. 30, 2023
(86) PCT No.: PCT/US2023/081803

§ 371 Date: Jan. 8, 2024

Related U.S. Application Data
(60) Provisional application No. 63/429,375, filed on Dec. 1, 2022.

(51) Int. Cl. B09B 3/60; B09B 101/85; C12N 1/14; C12R 1/645

(52) U.S. Cl. CPC B09B 3/60; C12N 1/14; B09B 2101/85; C12N 2500/76; C12R 2001/645

(57) ABSTRACT

Disclosed are methods for reducing contaminant levels in materials through remediation, including remediation using a filamentous fungus.

### RELATED APPLICATIONS

The present patent application claims the priority benefit of U.S. Provisional Patent Application Ser. No. 63/429,375, filed Dec. 1, 2022, the content of which is hereby incorporated by reference in its entirety into this disclosure.

### BACKGROUND

Mycoremediation is known to decontaminate toxic constituents in nature. Natural cleaners, fungi inhabit ecosystems present with heavy hydrocarbons. Fungi are widely regarded to be the best remediators of petroleum chemicals, more efficient than bacteria or other biological organisms. Fungi are capable of biodegrading petroleum hydrocarbons by secreting enzymes like laccases, tyrosinases, and manganese peroxidases to completely convert environmental toxins to non-threatening resources beneficial to the environment.

Per- and Polyfluoroalkyl Substances (PFAS), including perfluorooctanoic acids (PFOAs), are a large group of emerging environmental contaminants that are globally ubiquitous. These “forever chemicals” are highly recalcitrant and possess the capability to persist in the environment and accumulate in living organisms. The subject of human and environmental concern, the EPA issued a proposal Aug. 26, 2022, to designate two of the most widely used PFAS as hazardous substances under CERCLA (more specifically the Superfund program, the program established to address the cleanup of hazardous waste sites and respond to environmental emergencies). The rule would increase transparency around releases of these harmful chemicals and help to hold polluters accountable for cleaning up contamination.

Volatile Organic Compounds (VOCs) are another group of organic chemicals, known for easily evaporating into the air at room temperature, which may have short- or long-term adverse health effects. (e.g. trichloroethylene and vinyl chloride are known carcinogenic compounds.) In addition, emissions of VOCs to the outdoors are regulated by EPA mostly to prevent the formation of ozone, a constituent of photochemical smog. Semi-Volatile Organic Compounds (SVOCs) evaporate more slowly into the air and can be liquids or solids at room temperature. Some examples of products that include SVOCs are many pesticides, oil-based products, fire retardants, and some building materials. SVOCs can include polyaromatic hydrocarbons (PAHs), polychlorinated biphenyls (PCBs), polybrominated flame-retardants, and perfluoroalkyl acids (PFAS). Reducing the concentration of VOCs and SVOCs is an important health and environmental goal.

Additional research would validate the treatment to advance complete degradation of the contaminants or opportunities to fully mineralize all PFAS. Mycoremediation of the PFAS allows reuse of materials on site or into other applications, without posing future risk to human or environmental health.

### BRIEF SUMMARY

Using a novel mycoremidiation process, applicants of the present invention recently completed a study on gypsum fines, a waste product of construction and demolition. Applicants found that the mycoremediation process was highly effective in breaking down long chain fluorocarbons of PFAS, and thereby reducing levels of the harmful substance. A need exists for this mycoremidiation process as a solution to PFAS that would enable in situ treatments within a controlled environment. This would allow for environmental remediation processes to occur without being affected by weather conditions. Using filamentous fungi to degrade and sequester PFAS from contaminated sites, reducing soil and groundwater contamination and developing a platform for the fundamental research of the mycological degradation of these pollutants.

Disclosed herein are methods for reducing contaminant levels in at least one material, the method comprising inoculating the at least one material with a filamentous fungus to form an incubation mixture, wherein the filamentous fungus includes Gloeophyllum trabeum, Phanerochaete chrysosporium, Irpex lacteus, Lentinus crinitus, Pleurotus ostreatus, Ganoderma lucidum, Cunninghamella elegans, Trametes versicolor, Pseudeurotium sp, Geomyces sp, and/or a combination thereof; incubating the incubation mixture, wherein the incubation degrades a contaminant, wherein the contaminant is volatile organic compounds (VOCs), per- and polyfluoroalkyl substances (PFASs), and/or semi-volatile organic compounds (SVOCs), generated a treated material; and wherein the degradation of the contaminant results in reduced contaminant levels in the treated material.

In an aspect, the method results in reduced contaminant levels in the treated material of at least about 5%, alternatively by at least about 10%, alternatively by at least about 15%, alternatively by at least about 20%, alternatively by at least about 25%, alternatively by at least about 30%, alternatively by at least about 35%, alternatively by at least about 40%, alternatively by at least about 45%, alternatively by at least about 50%, alternatively by at least about 55%, alternatively by at least about 60%, alternatively by at least about 65%, alternatively by at least about 70%, alternatively by at least about 75%, alternatively by at least about 80%, alternatively by at least about 85%, alternatively by at least about 90%, alternatively by at least about 95%, alternatively by at least about 96%, alternatively by at least about 97%, alternatively by at least about 98%, alternatively by at least about 99%, or alternatively by at least about 100% when compared to an untreated material.

In an aspect, the at least one material is inoculated and/or incubated at a temperature of between about 40° F. to about 100° F., alternatively between about 50° F. to about 90° F., alternatively between about 60° F. to about 80° F., or alternatively between about 65° F. to about 75° F.

In an aspect, the material is inoculated at an inoculation rate of between about 2% and about 18% by weight of material, alternatively between about 5% and about 15% by weight of material, alternatively between about 8% and about 12% by weight of material, or alternatively between about 9% and about 11% by weight of material.

In an aspect, the incubation mixture is incubated for at least 24 hours, alternatively at least about three days, alternatively at least about one week, alternatively at least about two weeks, alternatively at least about three weeks, alternatively at least about four weeks, alternatively at least about at least five weeks, alternatively at least about six weeks, alternatively at least about seven weeks, alternatively at least about at least eight weeks, alternatively at least about twelve weeks.

In an aspect, the incubation mixture is incubated under aerobic conditions.

In an aspect, the material is mixed with a growth medium prior to inoculation and/or incubation.

In an aspect, growth medium comprises one or more of a lignin-containing material, sawdust, paper, cardboard, straw, wheat bran, hemp, dextrose, Light Malt Extract, gypsum, vermiculite, or sea minerals.

In an aspect, the material is processed prior to inoculation.

In an aspect, the processing comprises pulverizing the material until homogeneous and sterilizing the homogeneous material.

In an aspect, the processing further comprises screening the pulverized material for non-homogeneous pieces.

In an aspect, the method further comprises adding water to the material and/or to the growth media.

In an aspect, the method further comprises reducing or removing inhibitory components of the mixture that slow or prevent fungal growth.

In an aspect, the material is a building material. In an aspect, the building material comprises drywall, rubber, carpet, ceiling tiles, shingles, wood, insulation, and combinations thereof. In an aspect, the material is reusable in building construction after incubation.

In an aspect, the method further comprises sequestering the contaminant from the treated material.

In an aspect, the method results in a reduction in greenhouse gas emissions, reduces toxicity of the material, and/or creates a pathway for the reuse of the material.

One aspect of the disclosure is a method of reducing and/or preventing greenhouse gas emissions, the method comprising: onsite remediation of at least one building material, wherein the remediation comprises inoculating the at least one building material with a filamentous fungi to form an incubation mixture, wherein the filamentous fungi is selected from the group consisting of Gloeophyllum trabeum, Phanerochaete chrysosporium, Irpex lacteus, Lentinus crinitus, Lentinus tigrinus, Pleurotus ostreatus, Ganoderma lucidum, Cunninghamella elegans, Trametes versicolor, Pseudeurotium sp, Geomyces sp, and/or a combination thereof; incubating the incubation mixture, wherein the incubation degrades a contaminant, wherein the contaminant is volatile organic compounds (VOCs), per- and polyfluoroalkyl substances (PFASs), and/or semi-volatile organic compounds (SVOCs), generating a treated material, wherein the degradation of the contaminant results in reduced contaminant levels in the treated material; and wherein the onsite remediation reduces and/or prevents greenhouse gas emissions.

### BRIEF DESCRIPTION OF THE DRAWINGS

Various aspects of the present disclosure will now be described, by way of example only, with reference to the attached Figures, wherein:

FIG. 1 illustrates an example method of reducing contaminant levels materials shown as a diagram.

FIG. 2 are photographs from an exemplary use of the method on gypsum waste material.

FIG. 3 shows the results in a graph from an exemplary use of the method and the reduction of concentrations of certain PFAS long chain precursors.

FIG. 4 shows a graph of the concentrations of certain PFAS long chain precursors in an example sample of gypsum waste material treated with different fungi compared to a control

### DETAILED DESCRIPTION

Disclosed herein is are various method for reducing contaminant levels in materials using a mycological solution, in particular mycoremediation. Mycoremediation is a form of bioremediation in which fungi is used to degrade or isolate contaminants in materials. The methods disclosed include leveraging the fungal root structures (mycelium) to consume and eliminate toxins. In some methods filamentous fungi are used to degrade and/or sequester contaminants from various materials.

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

For any method disclosed herein that includes discrete steps, the steps may be conducted in any feasible order, and, as appropriate, any combination of two or more steps may be conducted simultaneously.

All headings are for the convenience of the reader and should not be used to limit the meaning of the text that follows the heading, unless so specified.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect,” “an aspect,” “certain aspects,” or “some aspects,” etc., means that a particular feature, configuration, composition, or characteristic described in connection with the aspect is included in at least one aspect of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

The invention is defined in the claims. However, below is a non-exhaustive listing of non-limiting exemplary aspects. Any one or more of the features of these aspects may be combined with any one or more features of another example, embodiment, or aspect described herein.

One aspect of the disclosure is a method for reducing contaminant levels in a least one material. In certain embodiments, the material is a building or construction material. Non-limiting examples include drywall, rubber, carpet, ceiling tiles, shingles, wood, insulation, and combinations of all of these materials. Building and constructions materials have been shown to have levels of contaminants, which include toxic chemicals such as per- and polyfluoroalkyl substances (PFASs), perfluorooctanoic acids (PFOAs), volatile organic compounds (VOCs), semi-volatile organic compounds (SVOCs), and precursors to these chemicals. In some embodiments PFAS longchain precursors include perfluoroalkyls, perfluorodecanesulfonic acid (PFDS), perfluorodecanoic acid (PFDA), perfluorododecanoic acid (PFDoA), perfluoroheptanesulfonic acid (PFHpS), perfluorohexanesulfonic acid (PFHxS), perfluorononanesulfonic acid (PFNS), perfluorodetradecanoic acid (PFTeA), perfluorotridecanoic acid (PFTriA), and perfluorundecanoic acid (PFUnA).

In an aspect, the method includes inoculating the material with a filamentous fungus to form an incubation mixture. As used herein “inoculation”, “inoculating”, and “inoculated” refer to the introduction of a fungus, including a filamentous fungus to a substrate. The fungus may further include mycelium. In some contexts, inoculation can involve introducing the fungus or fungi to the material to facilitate the reduction of contentment levels. In other contexts, inoculation can involve the introduction of fungi into a medium for growth or colonization. In general, incubation can refer to maintaining an environment for organism growth and biological processes.

A substrate may refer to materials or substances on which an organism acts. Depending on the embodiment, the material may be used as the substrate or a secondary material, such as growth medium, may be used as a substrate. The growth medium may provide necessary nutrients, water, catalysts for desired reactions, and/or physical support for growth of the filamentous fungi. Non-limiting examples of growth medium include a lignin-containing material, sawdust, paper, cardboard, straw, wheat bran, hemp, dextrose, Light Malt Extract (LME), gypsum, vermiculite, sea minerals, and combinations thereof. In some aspects, the growth medium in combined with the material prior to inoculation. In other aspects, the growth medium is added to the incubation mixture.

Filamentous fungi are characterized by filamentous, vegetative cells called hyphae. A mass of hyphae forms the thallus (vegetative body) of the fungus, which is composed of mycelium (Cole GT. Basic Biology of Fungi. In: Baron S, editor. Medical Microbiology. 4th edition. Galveston (TX): University of Texas Medical Branch at Galveston; 1996. Chapter 73. Available from: https://www.ncbi.nlm.nih.gov/books/NBK8099/). In some aspects, the filamentous fungi used to inoculate the material includes, but is not limited to, Gloeophyllum trabeum, Phanerochaete chrysosporium, Irpex lacteus, Lentinus crinitus, Pleurotus ostreatus, Ganoderma lucidum, Cunninghamella elegans, Trametes versicolor, Pseudeurotium sp, or Geomyces sp. In certain embodiments, the inoculation is with at least two filamentous fungi. In certain embodiments, the inoculation is with at least three filamentous fungi. In certain embodiments, the inoculation is with at least four filamentous fungi. In certain embodiments, the inoculation is with at least five filamentous fungi. In certain embodiments, the inoculation is with at least six filamentous fungi. In certain embodiments, the inoculation is with at least seven filamentous fungi. In certain embodiments, the inoculation is with at least eight filamentous fungi. In certain embodiments, the inoculation is with at least nine filamentous fungi. In certain embodiments, the inoculation is with at least ten filamentous fungi.

The resulting incubation mixture may include at least the material and the filamentous fungi. In certain embodiments, the incubation mixture may also include the growth material. Depending on the particular methods, the incubation mixture may include additional components. Non-limiting examples include a culture, a medium, and/or an inoculum. A culture may refer to a population of organisms that are growing under controlled conditions. A medium may refer to the materials, substance, or environment in which organisms grow. Inoculum may refer to material containing live organisms introduced into a medium to initiate a process, such as, but not limited to, growth,

In an aspect, the material is inoculated at a temperature of between about 40° F. to about 100° F. In certain embodiments, the material is inoculated at a temperature of between about 50° F. to about 90° F., alternatively between about 60° F. to about 80° F., or alternatively between about 65° F. to about 75° F. In certain embodiments, the material is inoculated at a temperature of about 40° F., alternatively about 45° F., alternatively about 50° F., alternatively about 55° F., alternatively about 60° F., alternatively about 65° F., alternatively about 70° F., alternatively about 75° F., alternatively about 80° F., alternatively about 85° F., alternatively about 90° F., alternatively about 95° F., or alternatively about 100° F.

In an aspect, the incubation mixture is incubated at a temperature of between about 40° F. to about 100° F. In certain embodiments, the incubation mixture is incubated at a temperature of between about 50° F. to about 90° F., alternatively between about 60° F. to about 80° F., or alternatively between about 65° F. to about 75° F. In certain embodiments, the incubation mixture is incubated at a temperature of about 40° F., alternatively about 45° F., alternatively about 50° F., alternatively about 55° F., alternatively about 60° F., alternatively about 65° F., alternatively about 70° F., alternatively about 75° F., alternatively about 80° F., alternatively about 85° F., alternatively about 90° F., alternatively about 95° F., or alternatively about 100° F.

In some aspects, the inoculation temperature and the incubation temperature are the same. In some aspects, the inoculation temperature and the incubation temperature are different.

In some aspects, the material is inoculated at an inoculation rate of between about 2% and about 18% by weight of material. In certain embodiments, the material is inoculated at an inoculation rate of between about 5% and about 15% by weight of material, alternatively between about 8% and about 12% by weight of material, or alternatively between about 9% and about 11% by weight of material. In certain embodiments, the material is inoculated at an inoculation rate of about 5% by weight of material, alternatively about 6% by weight of material, alternatively about 7% by weight of material, alternatively about 8% by weight of material, alternatively about 9% by weight of material, alternatively about 10% by weight of material, alternatively about 11% by weight of material, alternatively about 12% by weight of material, alternatively about 13% by weight of material, alternatively about 14% by weight of material, alternatively about 15% by weight of material, alternatively about 16% by weight of material, alternatively about 17% by weight of material, or alternatively about 18% by weight of material.

The time needed to incubate the incubation mixture may depend on several factors including, but not limited to, the species cultured, the volume and mass of the material, the desired product, the temperature, pH, and media used. In certain embodiments, the incubation time is at least about 24 hours. In certain embodiments, the incubation time is at least about 48 hours. In certain embodiments, the incubation time is at least about three days. In certain embodiments, the incubation time is at least about four days. In certain embodiments, the incubation time is at least about five days. In certain embodiments, the incubation time is at least about six days. In certain embodiments, the incubation time is at least about one week. In certain embodiments, the incubation time is at least about two weeks. In certain embodiments, the incubation time is at least about three weeks. In certain embodiments, the incubation time is at least about four weeks. In certain embodiments, the incubation time is at least about five weeks. In certain embodiments, the incubation time is at least about six weeks. In certain embodiments, the incubation time is at least about seven weeks. In certain embodiments, the incubation time is at least about eight weeks. In certain embodiments, the incubation time is at least about nine weeks. In certain embodiments, the incubation time is at least about ten weeks. In certain embodiments, the incubation time is at least about eleven weeks. In certain embodiments, the incubation time is at least about twelve weeks.

In an aspect, the incubation mixture is incubated under aerobic conditions. Aerobic conditions can refer to an environment or a process that involves, or requires, the presence of oxygen. It is in contrast to anaerobic conditions, where oxygen is either absent or present in very low concentrations.

In some aspects, the material may be processed before inoculation. In certain embodiments, the materials may be pulverized until homogenous. Breaking the material into smaller particles increases the surface area on which the fungi can act. The resulting homogenous material may then be sterilized. Sterilizing the material reduces and/or eliminates any contamination that may hinder or prevent the growth of fungi. In an aspect, the method can further include adding water to the material or to the growth media. In some embodiments, water may be needed for the survival of fungi and adding water to the material or the growth media can serve other purposes. In some aspects, the water can be distilled water, which can kill unwanted bacteria as well as dissolve substances in the materials and bring them into solution. In solution, the fungi can act on substances in the material that might not be accessible otherwise. In some aspects, contaminated water can be added to the material for a further reduction of contaminants.

In another aspect, the method may further include reducing or removing inhibitory components of the mixture the slow or prevent growth. For example, hydrogen sulfide is known to inhibit the growth of some fungal species. In certain embodiments, the method may include processes to reduce or remove hydrogen sulfide from the material. In an aspect, the method may include adding catalysts to materials to drive desired reactions. For example, laccasse is a copper-containing oxidase known in the art that requires the presence of copper.

The incubation as described unexpectedly reduces the contaminant levels, including the levels of VOCs, PFASs, and SVOCs in the material. In some aspects, the method reduces the levels of contaminants by at least about 5%, alternatively by at least about 10%, alternatively by at least about 15%, alternatively by at least about 20%, alternatively by at least about 25%, alternatively by at least about 30%, alternatively by at least about 35%, alternatively by at least about 40%, alternatively by at least about 45%, alternatively by at least about 50%, alternatively by at least about 55%, alternatively by at least about 60%, alternatively by at least about 65%, alternatively by at least about 70%, alternatively by at least about 75%, alternatively by at least about 80%, alternatively by at least about 85%, alternatively by at least about 90%, alternatively by at least about 95%, alternatively by at least about 96%, alternatively by at least about 97%, alternatively by at least about 98%, alternatively by at least about 99%, or alternatively by at least about 100% when compared to an untreated material.

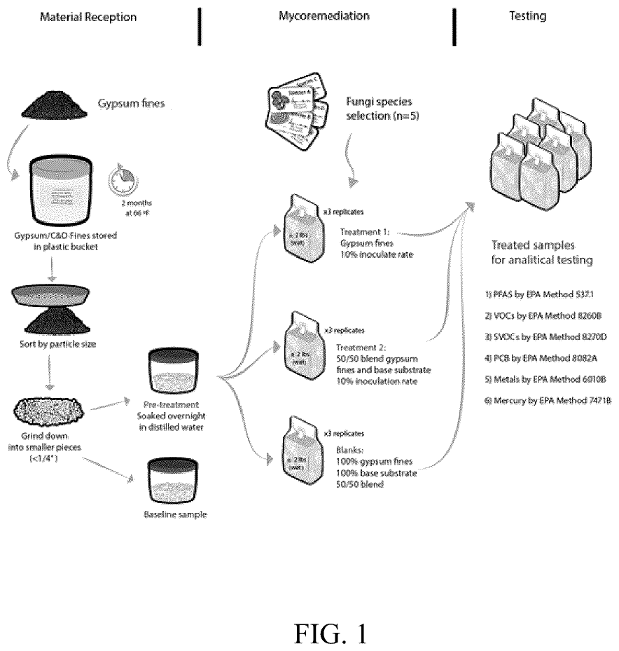

In an example embodiment, as shown in FIG. 1, the method was applied to gypsum fines and completed within four weeks with external environmental quality tests. In the example, the method demonstrated the reduction of PFAS and reduction across total petroleum hydrocarbons, phthalates and heavy metal sequestration.

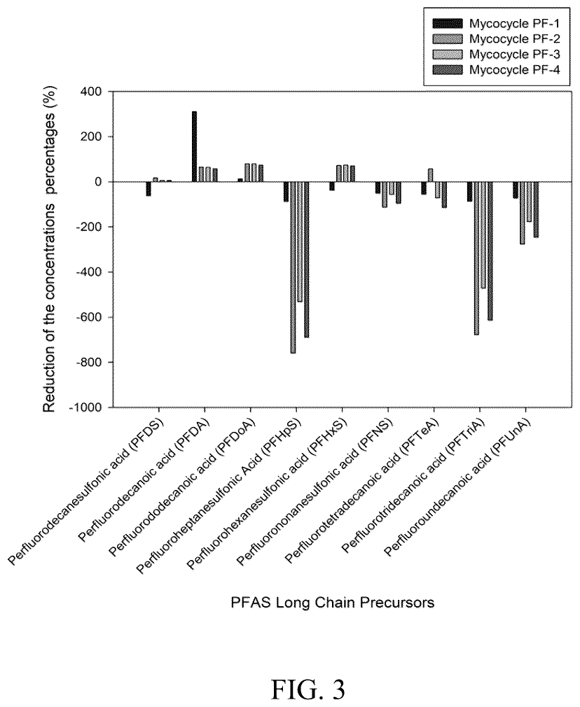

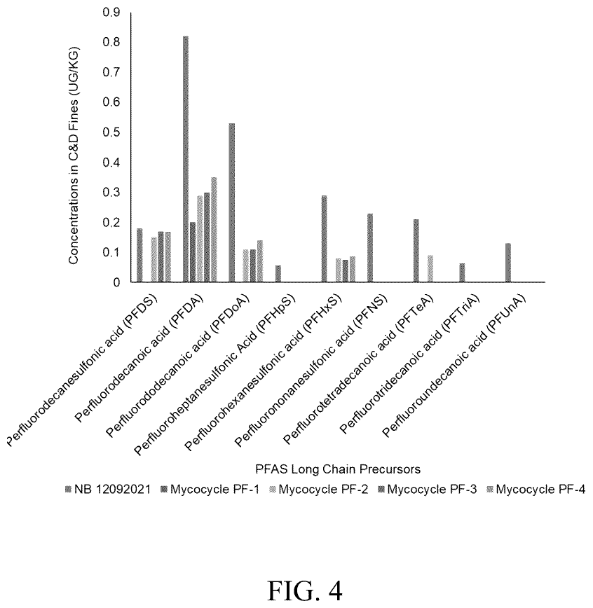

In another example embodiment, and as further described in Example 1 and as shown in FIG. 3, after application of the method there was a reduction of PFDA (75.6%) and PFDoA (11.32%) in Sample PF-1, a reduction of PFDS (16.6%), PFDA (64.6%), PFDOA (79%), PFHxS (72.4%), and PFTeA (56.6%) in Sample PF-2, a reduction of PFDS (5.5%), PFDA (63.4%), PFDoA (79%) and PFHxS (74.13%) in Sample PF-3, and a reduction of PFDS (5.5%), PFDA (57.3%), PFDoA (73.5%) and PFHxS (70%) in Sample PF-4 when compared with the control. As further supported by FIG. 4, when comparing a control with the treated samples, PFDA, PFHpSm PFNS, PFTriA and PFUnA were reduced in all samples, and PFDS, PFDoA, PHxS, and PFTcA were reduced in most others.

After incubation, the treated material has reduced levels contaminants while retaining at least some of the same properties that made the material useful. In certain embodiments, the treated material may be used or reused for its original purpose. In a non-limiting example, if the material used in the method was a building material, then the treated material may also be used in building construction.

In some aspects, the method further includes sequestering the contaminants. In certain embodiments, during and/or after incubation the contaminants may be captured and physically removed from the environment. Various sequestering techniques including, but not limited to, linear alkylsilane phase (C4, C8, C18) chromatography, gas chromatography and liquid-liquid extraction, may be used to sequester the contaminants. In some aspects, the sequestered contaminants are then destroyed using degradation techniques. Non-limiting examples suitable degradation techniques include decarboxylation, desulfonation, and/or defluorination.

By combining the disclosed methods with sequestering and degradation techniques may result in a complete or substantially complete removal of these contaminants from the environments. For example, in certain aspects, the method reduces containments from various waste channels, reduces greenhouse gas emissions, including those related to transporting and disposal of materials, reduces toxicity of building waste materials, and/or create pathways for reuse of building materials. The method also achieves a reduction of carbon costs in waste, transport, and/or manufacturing of building materials.

The disclosed methods may be implemented across a variety of materials and contaminants. In some aspects, the methods may be mobile and setup in an isolated, contained environment. In some aspects, the methods may can be implemented with materials across the construction sector with microprocessing sites to decentralize waste management and encourage large-scale adoption of the innovative technology. Options for large scale treatment are available through the bioremediation specialists, composters, waste remediators and recyclers-all with specific knowledge and/or equipment—could be trained to implement the method on-site or apply the process in situ. An embodiment of the method may also be employed in a controlled ambient environment such as an intermodal container outfitted with an air conditioner.

One aspect of the disclosure includes a method of reducing and/or preventing greenhouse gas emissions through on-site remediation. In conventional methods, materials, such as building materials, that can't be reused on-site are transported to a landfill or recycling facility. These materials can also contain contaminants and the disposal process may be challenging. According to the US Environmental Protection Agency, transportation generates the largest share of greenhouse gas emissions (28% of 2021 greenhouse gas emissions) https://www.epa.gov/ghgemissions/sources-greenhouse-gas-emissions. By allowing for on-site remediation, for example using a mobile, isolated, contained environment, the inventors have unexpectantly discovered a method for reducing greenhouse gas emissions while also reducing contaminants. In addition, reuse of the building materials saves the carbon costs of manufacturing.

The presently described technology and its advantages will be better understood by reference to the following examples. These examples are provided to describe specific implementations of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### EXAMPLES

### Example 1: PFAS Reduction from Gypsum Waste

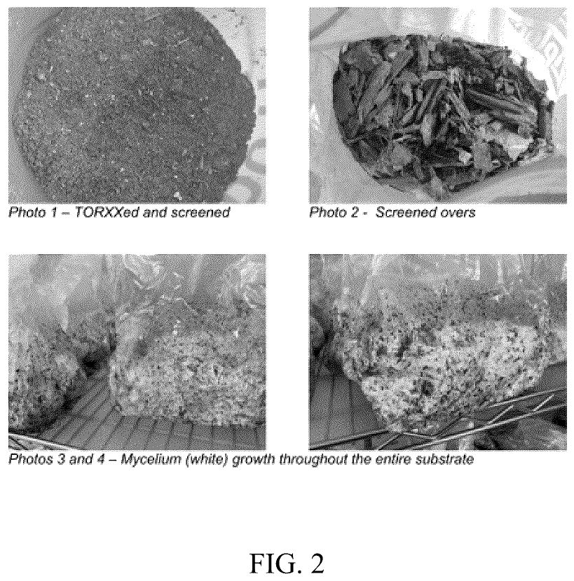

Gypsum fines were ground down with a TORXX Kinetic Pulverizer. The left upper panel of FIG. 2 shows pulverized and homogenized material prior to inoculation. These were stored for two months in buckets at 66° F. and later sorted by particle size, only pieces of substrate smaller than ¼″ were used. The right upper panel photograph of FIG. 2 shows screened pieces of gypsum that were deemed too large for the culture.

Fines were soaked overnight with distilled water to hydrate and kill any potential microorganisms. Gypsum fines were then mixed with inoculum and sterilized for inoculation. Two substrate formulations were used: 100% sorted gypsum fines and a 50/50 blend of gypsum fines with medium. Inoculation rates with treatment spawn remained the same (10% by weight) throughout the study.

The pre-treated gypsum fines were inoculated with five fungal strains. The treated blends were allowed to incubate for three weeks under optimal environmental conditions. In the treated samples were sent to a third-party analytical testing lab. Photographs of the colonized material are presented in the lower two panels of FIG. 2.

Analysis of the gypsum blank (gypsum without fungal colonization) included testing for concentrations of semi-volatile organic compounds, organic compounds, heavy metals and long chain PFOS and PFOAS.

### Example 2: Analytical Results of Long Chain Perfluoroalkylated Compounds

Reductions in PFAS concentrations between the baseline sample and the four treated samples were calculated using the formula (below) to determine reduction or increment of the compound concentrations on the solid samples. Compounds not detectable during testing were omitted.

(n=(a−b)/a*100))
(n is % reduction, a is initial concentration, b is final concentration)

FIG. 3 presents a graph comparing the gypsum blank and the four Mycocycle samples (PF-1, PF-2, PF-3 & PF-4) showing the reduction on the concentration percentages of the compounds. The graph presents percent reduction of the concentration perfluoroalkylated compounds by percentages per sample compared to baseline sample. The results of the exemplary study show a reduction of PFDA (75.6%) and PFDoA (11.32%) in Sample PF-1; a reduction of PFDS (16.6%), PFDA (64.6%), PFDoA (79%), PFHxS (72.4%), and PFTeA (56.6%) in Sample PF-2; a reduction of PFDS (5.5%), PFDA (63.4%), PFDoA (79%) and PFHxS (74.13) in Sample PF-3; a reduction of PFDS (5.5%), PFDA (57.3%), PFDoA (73.5%) and PFHxS (70%) in Sample PF-4 when compared with the control.

When comparing gypsum blank and the four Mycocycle samples (PF-1, PF-2, PF-3 & PF-4) some compounds showed reductions as shown in FIG. 4. FIG. 4 presents a graph showing a reduction of the long chain perfluoroalkylated compound by sample. Each sample displays a different color. Comparing the blank sample compound with the other four samples PFDS, PFDoA and PHxS were reduced in all samples except for PF-1. PFTeA were reduced in all samples except for PF-2. PFDA, PFHpSm PFNS, PFTriA and PFUnA were reduced in all samples.

### Example 3: VOCs/SOCs Reduction from Gypsum Waste

When comparing gypsum blank and the four Mycocycle samples some compounds, the levels of POCs are reduced with few outlying exceptions. Table 1 shows the levels of POCs reduction in four treated samples compared to the blank (NB 12092021). Table 2 shows the levels of SPOCs reduction in four treated samples compared to the blank (NB 12092021).

TABLE 1

| | NB 12092021 | Mycocycle-1 | Mycocycle-2 | Mycocycle-3 | Mycocycle-4 |
|---|---|---|---|---|---|
| 1,1,1-Trichloroethane | 0.13 | 0.0052 | 0.0049 | 0.0050 | 0.0037 |
| 1,1,2,2-Tetrachloroethane | 0.13 | 0.0052 | 0.0049 | 0.0050 | 0.0037 |
| 1,1,2-Trichloroethane | 0.13 | 0.0052 | 0.0049 | 0.0050 | 0.0037 |
| 1,1-Dichloroethane | 0.13 | 0.0052 | 0.0049 | 0.0050 | 0.0037 |
| 1,1-Dichloroethene | 0.20 | 0.0052 | 0.0049 | 0.0050 | 0.0037 |
| 1,2-Dichloroethane | 0.13 | 0.013 | 0.012 | 0.012 | 0.0093 |
| 1,2-Dichloropropane | 0.13 | 0.0052 | 0.0049 | 0.0050 | 0.0037 |
| 1,3-Dichloropropene, Total | 0.13 | 0.0052 | 0.0049 | 0.0050 | 0.0037 |
| 2-Butanone (MEK) | 0.66 | 0.13 | 0.0098 | 0.015 | 0.0093 |
| 2-Hexanone | 0.66 | 0.013 | 0.012 | 0.012 | 0.0093 |
| Acetone | 1.3 | 7.7 | 0.37 | 5.4 | 0.10 |
| Benzene | 0.033 | 0.0052 | 0.0049 | 0.0050 | 0.0037 |
| Bromodichloromethane | 0.13 | 0.0052 | 0.0049 | 0.0050 | 0.0037 |
| Bromoform | 0.13 | 0.0052 | 0.0049 | 0.0050 | 0.0037 |
| Bromomethane | 0.39 | 0.013 | 0.012 | 0.012 | 0.0093 |
| Carbon disulfide | 0.26 | 0.013 | 0.012 | 0.012 | 0.0093 |
| Carbon tetrachloride | 0.13 | 0.0052 | 0.0049 | 0.0050 | 0.0037 |
| Chlorobenzene | 0.13 | 0.0052 | 0.0049 | 0.0050 | 0.0037 |
| Chloroethane | 0.13 | 0.013 | 0.012 | 0.012 | 0.0093 |
| Chloroform | 0.26 | 0.0052 | 0.0049 | 0.0050 | 0.0037 |
| Chloromethane | 0.13 | 0.013 | 0.0094 | 0.012 | 0.0093 |
| cis-1,2-Dichloroethene | 0.13 | 0.0052 | 0.0049 | 0.0050 | 0.0037 |
| cis-1,3-Dichloropropene | 0.13 | 0.0052 | 0.0049 | 0.0050 | 0.0037 |
| Dibromochloromethane | 0.13 | 0.0052 | 0.0049 | 0.0050 | 0.0037 |
| Ethylbenzene | 0.033 | 0.0052 | 0.0049 | 0.0050 | 0.0037 |
| methyl isobutyl ketone | 0.66 | 0.013 | 0.012 | 0.012 | 0.0093 |
| Methyl tert-butyl ether | 0.13 | 0.0052 | 0.0049 | 0.0050 | 0.0037 |
| Methylene Chloride | 0.66 | 0.013 | 0.012 | 0.012 | 0.0093 |
| Styrene | 0.13 | 0.0052 | 0.0049 | 0.0050 | 0.0037 |
| Tetrachloroethene | 0.13 | 0.0052 | 0.0049 | 0.0024 | 0.0037 |
| Toluene | 0.14 | 0.0052 | 0.0012 | 0.0050 | 0.0037 |
| trans-1,2-Dichloroethene | 0.13 | 0.0052 | 0.0049 | 0.0050 | 0.0037 |
| trans-1,3-Dichloropropene | 0.13 | 0.0052 | 0.0049 | 0.0050 | 0.0037 |
| Trichloroethene | 0.066 | 0.0052 | 0.0049 | 0.0050 | 0.0037 |
| Vinyl chloride | 0.13 | 0.0052 | 0.0049 | 0.0050 | 0.0037 |
| Xylenes, Total | 0.17 | 0.0041 | 0.0033 | 0.013 | 0.0075 |

TABLE 2

| | NB 12092021 | Mycocycle-1 | Mycocycle-2 | Mycocycle-3 | Mycocycle-4 |
|---|---|---|---|---|---|
| 1,2,4-Trichlorobenzene | 12 | 3.0 | 2.8 | 3.0 | 2.3 |
| 1,2-Dichlorobenzene | 12 | 3.0 | 2.8 | 3.0 | 2.3 |
| 1,3-Dichlorobenzene | 12 | 3.0 | 2.8 | 3.0 | 2.3 |
| 1,4-Dichlorobenzene | 12 | 3.0 | 2.8 | 3.0 | 2.3 |
| 2,2′-oxybis[1-chloropropane] | 12 | 3.0 | 2.8 | 3.0 | 2.3 |
| 2,4,5-Trichlorophenol | 23 | 5.9 | 5.6 | 5.9 | 4.5 |
| 2,4,6-Trichlorophenol | 23 | 5.9 | 5.6 | 5.9 | 4.5 |
| 2,4-Dichlorophenol | 23 | 5.9 | 5.6 | 5.9 | 4.5 |
| 2,4-Dimethylphenol | 23 | 5.9 | 5.6 | 5.9 | 4.5 |
| 2,4-Dinitrophenol | 47 | 12 | 11 | 12 | 9.2 |
| 2,4-Dinitrotoluene | 12 | 3.0 | 2.8 | 3.0 | 2.3 |
| 2,6-Dinitrotoluene | 12 | 3.0 | 2.8 | 3.0 | 2.3 |
| 2-Chloronaphthalene | 12 | 3.0 | 2.8 | 3.0 | 2.3 |
| 2-Chlorophenol | 12 | 3.0 | 2.8 | 3.0 | 2.3 |
| 2-Methylnaphthalene | 0.79 | 1.7 | 0.84 | 0.53 | 0.30 |
| 2-Methylphenol | 12 | 3.0 | 2.8 | 3.0 | 2.3 |
| 2-Nitroaniline | 12 | 3.0 | 2.8 | 3.0 | 2.3 |
| 2-Nitrophenol | 23 | 5.9 | 5.6 | 5.9 | 4.5 |
| 3 & 4 Methylphenol | 12 | 3.0 | 2.8 | 3.0 | 2.3 |
| 3,3′-Dichlorobenzidine | 12 | 3.0 | 2.8 | 3.0 | 2.3 |
| 3-Nitroaniline | 23 | 5.9 | 5.6 | 5.9 | 4.5 |
| 4,6-Dinitro-2-methylphenol | 47 | 12 | 11 | 12 | 9.2 |
| 4-Bromophenyl phenyl ether | 12 | 3.0 | 2.8 | 3.0 | 2.3 |
| 4-Chloro-3-methylphenol | 23 | 5.9 | 5.6 | 5.9 | 4.5 |
| 4-Chloroaniline | 47 | 12 | 11 | 12 | 9.2 |
| 4-Chlorophenyl phenyl ether | 12 | 3.0 | 2.8 | 3.0 | 2.3 |
| 4-Nitroaniline | 23 | 5.9 | 5.6 | 5.9 | 4.5 |
| 4-Nitrophenol | 47 | 12 | 11 | 12 | 9.2 |
| Acenaphthene | 0.42 | 18 | 9.0 | 6.3 | 3.1 |
| Acenaphthylene | 1.4 | 0.24 | 0.16 | 0.11 | 0.085 |
| Anthracene | 2.2 | 29 | 18 | 13 | 9.4 |
| Benzo[a] anthracene | 5.6 | 85 | 52 | 42 | 32 |
| Benzo[a]pyrene | 6.5 | 86 | 56 | 46 | 34 |
| Benzo[b]fluoranthene | 8.3 | 100 | 65 | 54 | 43 |
| Benzo[g,h,i]perylene | 1.7 | 48 | 36 | 26 | 18 |
| Benzo[k]fluoranthene | 4.2 | 41 | 26 | 22 | 16 |
| Bis(2-chloroethoxy)methane | 12 | 3.0 | 2.8 | 3.0 | 2.3 |
| Bis(2-chloroethyl)ether | 12 | 3.0 | 2.8 | 3.0 | 2.3 |
| Bis(2-ethylhexyl) phthalate | 4.4 | 1.4 | 2.8 | 3.0 | 2.3 |
| Butyl benzyl phthalate | 12 | 3.0 | 2.8 | 3.0 | 2.3 |
| Carbazole | 12 | 16 | 8.7 | 5.2 | 4.5 |
| Chrysene | 6.0 | 93 | 56 | 46 | 31 |
| Dibenz(a,h)anthracene | 0.71 | 11 | 7.8 | 6.2 | 4 |
| Dibenzofuran | 12 | 11 | 4.7 | 3.6 | 2.0 |
| Diethyl phthalate | 12 | 3.0 | 2.8 | 3.0 | 2.3 |
| Dimethyl phthalate | 12 | 3.0 | 2.8 | 3.0 | 2.3 |
| Di-n-butyl phthalate | 12 | 3.0 | 2.8 | 3.0 | 2.3 |
| Di-n-octyl phthalate | 12 | 3.0 | 2.8 | 3.0 | 2.3 |
| Fluoranthene | 14 | 260 | 150 | 120 | 76 |
| Fluorene | 1.3 | 17 | 8.9 | 6.0 | 3.6 |
| Hexachloro-1,3-butadiene | 12 | 3.0 | 2.8 | 3.0 | 2.3 |
| Hexachlorobenzene | 4.7 | 1.2 | 1.1 | 1.2 | 0.92 |
| Hexachlorocyclopentadiene | 47 | 12 | 11 | 12 | 9.2 |
| Hexachloroethane | 12 | 3.0 | 2.8 | 3.0 | 2.3 |
| Indeno[1,2,3-cd]pyrene | 1.8 | 45 | 31 | 25 | 16 |
| Isophorone | 12 | 3.0 | 2.8 | 3.0 | 2.3 |
| Naphthalene | 2.4 | 5.9 | 2.3 | 1.8 | 0.70 |
| Nitrobenzene | 2.3 | 0.59 | 0.56 | 0.59 | 0.45 |
| N-Nitrosodi-n-propylamine | 4.7 | 1.2 | 1.1 | 1.2 | 0.92 |
| N-Nitrosodiphenylamine | 12 | 3.0 | 2.8 | 3.0 | 2.3 |
| Pentachlorophenol | 47 | 12 | 11 | 12 | 9.2 |
| Phenanthrene | 10 | 200 | 91 | 76 | 48 |
| Phenol | 12 | 3.0 | 2.8 | 3.0 | 2.3 |
| Pyrene | 12 | 210 | 120 | 98 | 68 |

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for reducing contaminant levels in at least one material, the method comprising:
inoculating the at least one material with a filamentous fungus to form an incubation mixture, wherein the filamentous fungi is selected from the group consisting of Gloeophyllum trabeum, Phanerochaete chrysosporium, Irpex lacteus, Lentinus crinitus, Lentinus tigrinus, Pleurotus ostreatus, Ganoderma lucidum, Cunninghamella elegans, Trametes versicolor, Pseudeurotium sp, Geomyces sp, and/or a combination thereof;
incubating the incubation mixture, wherein the incubation degrades a contaminant, wherein the contaminant is volatile organic compounds (VOCs), per- and polyfluoroalkyl substances (PFASs), and/or semi-volatile organic compounds (SVOCs), generating a treated material,
and wherein the degradation of the contaminant results in reduced contaminant levels in the treated material.

2. The method of claim 1, wherein the method results in reduced contaminant levels in the treated material of at least about 5% when compared to an untreated material.

3. The method of claim 1, wherein the at least one material is inoculated and/or incubated at a temperature of between about 40° F. to about 100° F.

4. The method of claim 1, wherein the material is inoculated at an inoculation rate of between about 2% and about 18% by weight of material.

5. The method of claim 1, wherein the incubation mixture is incubated for at least 24 hours.

6. The method of claim 1, wherein the incubation mixture is incubated under aerobic conditions.

7. The method of claim 1, wherein the material is mixed with a growth medium prior to inoculation and/or incubation.

8. The method of claim 7, wherein the growth medium comprises one or more of a lignin-containing material, sawdust, paper, cardboard, straw, wheat bran, hemp, dextrose, Light Malt Extract, gypsum, vermiculite, or sea minerals.

9. The method of claim 8, wherein the at least one material is processed prior to inoculation.

10. The method of claim 9, wherein the processing comprises pulverizing the at least one material until homogeneous and sterilizing the homogeneous material.

11. The method of claim 10, wherein the processing further comprises screening the pulverized material for non-homogeneous pieces.

12. The method of claim 1, further comprising adding water to the material and/or to the growth medium.

13. The method of claim 1, further comprising reducing and/or removing inhibitory components of the mixture that slow or prevent fungal growth.

14. The method of claim 1, wherein the material is a building material.

15. The method of claim 14, wherein the building material comprises drywall, rubber, carpet, ceiling tiles, shingles, wood, insulation, and combinations thereof.

16. The method of claim 14, wherein the treated material is usable in building construction.

17. The method of claim 1, further comprising sequestering and/or destroying the contaminant.

18. The method of claim 1, wherein the method results in a reduction in greenhouse gas emissions, reduces toxicity of the material, and/or creates a pathway for the use of the treated material.

19. A method of reducing and/or preventing greenhouse gas emissions, the method comprising:
on-site remediation of at least one building material, wherein the remediation comprises
inoculating the at least one building material with a filamentous fungus to form an incubation mixture, wherein the filamentous fungi fungus is selected from the group consisting of Gloeophyllum trabeum, Phanerochaete chrysosporium, Irpex lacteus, Lentinus crinitus, Lentinus tigrinus, Pleurotus ostreatus, Ganoderma lucidum, Cunninghamella elegans, Trametes versicolor, Pseudeurotium sp, Geomyces sp, and/or a combination thereof;
incubating the incubation mixture, wherein the incubation degrades a contaminant, wherein the contaminant is volatile organic compounds (VOCs), per -and polyfluoroalkyl substances (PFASs), and/or semi-volatile organic compounds (SVOCs), generating a treated material, wherein the degradation of the contaminant results in reduced contaminant levels in the treated material; and
wherein the onsite remediation reduces and/or prevents greenhouse gas emissions.
